(19) United States
(12) Patent Application Publication
(10) Pub. No.: US 2023/0210865 A1
(43) Pub. Date: Jul. 6, 2023

(54) MIDAZOLAM PREMIX FORMULATIONS AND USES THEREOF

(71) Applicants: Baxter International, Inc., Deerfield, IL (US); Baxter Healthcare SA, Glattpark (Opfikon) (CH)

(72) Inventors: Chiragkumar Girishbhai Siddhapara, Rajkot (IN); Piyushkumar Amrutlal Patel, Ahmedabad (IN); Pankajkumar Ramabhai Patel, Ahmedabad (IN)

(21) Appl. No.: 18/146,499

(22) Filed: Dec. 27, 2022

(30) Foreign Application Priority Data
Dec. 29, 2021 (IN) 202141061523

(51) Int. Cl. A61K 31/5517; A61K 47/02; A61J 1/14

(52) U.S. Cl. CPC A61K 31/5517; A61K 47/02; A61J 1/1468

(57) ABSTRACT

The present disclosure is directed to a midazolam pharmaceutical premix formulation housed in a flexible container, comprising from about 0.4 mg/ml to about 1.1 mg/ml midazolam hydrochloride and sodium chloride, wherein the formulation has a pH from about 2.7 to about 3.5. Also disclosed herein are methods of sedating a human subject in need thereof using the premix formulation.

### RELATED APPLICATIONS

This application claims priority to Indian application no. 202141061523, filed on Dec. 29, 2021. The contents of the foregoing application are hereby incorporated by reference herein.

### TECHNICAL FIELD

The present disclosure generally relates to stable liquid midazolam pharmaceutical premix formulations. The present disclosure also relates to methods of using said liquid midazolam pharmaceutical premix formulations.

### BACKGROUND

Midazolam hydrochloride (HCl) is a short-acting benzodiazepine central nervous system (CNS) depressant used in anesthesia. The effects of midazolam HCl on the CNS are generally dependent on the dose administered, the route of administration, and the presence or absence of other medications. Midazolam HCl, administered by injection, is a potent sedative agent having about twice the potency of the classical benzodiazepine anesthesia agent diazepam. Onset time of sedative effects after intramuscular administration in adults is about fifteen minutes, with peak sedation occurring within 30 to 60 minutes following injection.

Midazolam HCl injection generally requires slow administration and individualization of dosage. Excessive single doses or rapid intravenous administration may result in respiratory depression, airway obstruction and/or arrest. Commonly midazolam is provided in vials which requires dilution and preparation prior to administration. Such additional steps can provide error—resulting in negative consequences for the patient. Thus, safer and more efficient means for delivering midazolam are needed.

### SUMMARY

Typically, midazolam hydrochloride (HCl) must be diluted prior to administration to a patient. The requisite dilution is inconvenient, time consuming, and can be subject to errors and product contamination. Therefore, a need exists for an improved midazolam formulation that is stable, and contains an appropriate premix amount of midazolam HCl such that the formulation is ready for delivery to the patient. Described herein are midazolam HCl formulations that are premixed and ready for infusion to the patient. The premix formulation described herein is preferably in a flexible, plastic container, e.g., a bag, which is fabricated from a multilayer sheeting composed of polypropylene, polyamide, and polyethylene. In certain embodiments, the innermost layer which is in contact with the formulation is made of polyethylene.

Provided herein is a pharmaceutical premix formulation comprising about 0.4 milligrams per milliliter (mg/ml) to about 1.1 mg/ml midazolam hydrochloride (HCl) and sodium chloride, wherein the formulation is an aqueous, premix formulation and has a pH from about 2.5 to about 3.7.

In one embodiment, the pharmaceutical premix formulation comprises from about 8.5 mg/ml to about 9.5 mg/ml sodium chloride. In one embodiment, the pharmaceutical premix formulation comprises about 9.0 mg/ml sodium chloride. In another embodiment, the pharmaceutical premix formulation comprises about 0.9% sodium chloride.

In certain embodiments, the pharmaceutical premix formulation has a pH from about 2.5 to about 3.7. In other embodiments, the pharmaceutical premix formulation has a pH from about 2.8 to about 3.4. In still other embodiments, the pharmaceutical premix formulation has a pH of about 3.0.

In one embodiment, the pharmaceutical premix formulation comprises about 0.5 mg/ml midazolam HCl. In another embodiment, the pharmaceutical premix formulation comprises about 1 mg/ml midazolam HCl. In still another embodiment, the pharmaceutical premix formulation comprises from about 50 to about 100 mg of midazolam HCl.

Provided herein is also a pharmaceutical premix formulation in a flexible plastic container, comprising from about 0.4 mg/ml to about 1.1 mg/ml midazolam hydrochloride (HCl) and sodium chloride, wherein the formulation is an aqueous, premix formulation and has a pH of from about 2.5 to about 3.7; and the flexible plastic container is fabricated from a multilayer sheeting comprising polypropylene, polyamide, and polyethylene.

In one embodiment, the pharmaceutical premix formulation comprises from about 8.5 mg/mi to about 9.5 mg/ml sodium chloride. In one embodiment, the pharmaceutical premix formulation comprises from about 9.0 mg/ml sodium chloride. In one embodiment, the pharmaceutical premix formulation comprises about 0.9% sodium chloride. In one embodiment, the pharmaceutical premix formulation has a pH from about 2.6 to about 3.6.

In one embodiment, the pharmaceutical premix formulation has a pH from about 2.8 to about 3.4. In one embodiment, the pharmaceutical premix formulation has a pH from about 2.50 to about 2.95. In one embodiment, the pharmaceutical premix formulation comprises about 0.5 mg/ml midazolam HCl. In one embodiment, the pharmaceutical premix formulation comprises about 1 mg/ml midazolam HCl. In one embodiment, the pharmaceutical premix formulation comprises 50 to about 100 mg of midazolam HCl.

Also provided herein is a pharmaceutical premix formulation comprising from about 50 to about 100 mg of midazolam HCl at a concentration from about 0.5 mg/ml to about 1.0 mg/ml, and from about 8.4 mg/ml to about 9.4 mg/ml sodium chloride, wherein the formulation is an aqueous, premix formulation and has a pH from about 2.5 to about 3.7. In one embodiment, the pharmaceutical premix formulation comprises about 9 mg/ml sodium chloride.

Also provided herein is a pharmaceutical premix formulation comprising about 50 mg of midazolam HCl at a concentration of about 0.5 mg/ml, and about 0.9% sodium chloride, wherein the formulation is an aqueous, premix formulation and has a pH from about 2.5 to about 3.7.

Also provided herein is a pharmaceutical premix formulation comprising about 100 mg of midazolam HCl at a concentration of about 1.0 mg/ml, and about 0.9% sodium chloride, wherein the formulation is an aqueous, premix formulation and has a pH from about 2.5 to about 3.7.

Provided herein is also a pharmaceutical premix formulation in a flexible plastic container, comprising from about 50 to about 100 mg of midazolam HCl at a concentration from about 0.5 mg/ml to about 1.0 mg/ml, and from about 8.4 mg/ml to about 9.4 mg/ml sodium chloride, wherein the formulation is an aqueous, premix formulation and has a pH from about 2.5 to about 3.7; and the flexible plastic container is fabricated from a multilayer sheeting comprising polypropylene, polyamide, and polyethylene. In one embodiment, the pharmaceutical premix formulation comprises about 9 mg/ml sodium chloride.

Also provided herein is a pharmaceutical premix formulation in a flexible plastic container, comprising about 50 mg of midazolam HCl at a concentration of about 0.5 mg/ml, and about 0.9% sodium chloride, wherein the formulation is an aqueous, premix formulation and has a pH from about 2.5 to about 3.7; and the flexible plastic container is fabricated from a multilayer sheeting comprising polypropylene, polyamide, and polyethylene.

Further provided herein is a pharmaceutical premix formulation in a flexible plastic container, comprising 100 mg of midazolam HCl at a concentration of about 1.0 mg/ml, and about 0.9% sodium chloride, wherein the formulation is an aqueous, premix formulation, has a pH of from about 2.5 to about 3.7; and the flexible plastic container is fabricated from a multilayer sheeting comprising polypropylene, polyamide, and polyethylene.

In one embodiment, the pharmaceutical premix formulation has a pH from about 2.50 to about 2.95.

In another embodiment, the pharmaceutical premix formulation is essentially free of a preservative.

Further provided herein is a pharmaceutical premix formulation consisting essentially of about 50 mg or about 100 mg of midazolam HCl at a concentration of about 1.0 mg/ml of midazolam, and about 0.9% sodium chloride, wherein the pharmaceutical premix formulation is an aqueous formulation having a pH from about 2.5 to about 3.7.

Also provided herein is a pharmaceutical premix formulation consisting essentially of about 25 mg or about 50 mg of midazolam HCl at a concentration of about 0.5 mg/ml, and about 0.9% sodium chloride, wherein the pharmaceutical premix formulation is an aqueous formulation having a pH from about 2.5 to about 3.7.

Described herein is also a pharmaceutical premix formulation in a flexible plastic container, the formulation consisting essentially of 100 mg of midazolam HCl at a concentration of about 1.0 mg/ml, and about 0.9% sodium chloride; or about 50 mg of midazolam HCl at a concentration of about 0.5 mg/ml, and about 0.9% sodium chloride, wherein, the pharmaceutical premix formulation is an aqueous, premix formulation and has a pH from about 2.5 to about 3.7; and the flexible plastic container is fabricated from a multilayer sheeting comprising polypropylene, polyamide, and polyethylene.

In one embodiment, the pharmaceutical premix formulation is stable for at least 6 months at about 25 degrees Celsius.

In one embodiment, the pharmaceutical premix formulation is not diluted prior to intravenous infusion into a subject.

Also provided herein is a flexible container comprising the pharmaceutical premix formulation of disclosed herein. In one embodiment, the flexible container consists essentially of a pharmaceutical premix formulation described herein.

In one embodiment, the flexible container is a bag. In one embodiment, the flexible container is polypropylene (PP), polyamide (PA), polyethylene (PE), or a combination thereof. In one embodiment, the inner layer of the flexible container is polyethylene. In another embodiment, the flexible, plastic container has a volume of about 50 mL or about 100 mL.

Also provided is a method of sedating a human subject in need thereof, the method comprising administering a pharmaceutical premix formulation disclosed herein to the human subject.

Further provided is a method of sedating a human subject in need thereof, the method comprising obtaining a flexible container comprising a pharmaceutical premix formulation described herein, placing the flexible container on a hook or means for suspending the flexible container, and intravenously administering the pharmaceutical premix formulation to the human subject.

Also provided herein is a method of sedating a human subject in need thereof, the method comprising intravenously administering to the human subject in need of sedation a pharmaceutical premix formulation comprising from about 0.5 mg/ml to about 1 mg/mi midazolam HCl and from about 0.8% to about 0.9% sodium chloride, wherein the pharmaceutical premix formulation is an aqueous formulation which is not diluted prior to administration to the human subject.

Also provided is a method of sedating a human subject in need thereof, the method comprising a) obtaining a flexible plastic container which is fabricated from a multilayer sheeting comprising polypropylene, polyamide, and polyethylene, wherein the flexible plastic container comprises a pharmaceutical premix formulation consisting essentially of about 100 mg of midazolam HCl at a concentration of about 1.0 mg/ml, and about 0.9% sodium chloride, or about 50 mg of midazolam HCl at a concentration of about 0.5 mg/ml, and about 0.9% sodium chloride, wherein the pharmaceutical premix formulation is an aqueous formulation and has a pH from about 2.5 to about 3.7, b) placing the flexible plastic container on a hook or means for suspending the flexible plastic container, and c) intravenously administering the pharmaceutical premix formulation to the human subject.

Described herein is also a method of sedating a human subject in need thereof, the method comprising intravenously administering to the human subject in need of sedation, a pharmaceutical premix formulation comprising from about 0.5 mg/ml to about 1 mg/ml midazolam HCl and from about 0.8% to about 0.9% sodium chloride, wherein the pharmaceutical premix formulation is an aqueous, premix formulation housed in a plastic flexible container which is fabricated from a multilayer sheeting comprising polypropylene, polyamide, and polyethylene.

In one embodiment, the pharmaceutical premix formulation comprises about 25 mg or about 50 mg midazolam HCl at a concentration of about 0.5 mg/ml. In one embodiment, the pharmaceutical premix formulation comprises about 50 mg or about 100 mg midazolam HCl at a concentration of about 1 mg/ml. In one embodiment, the pharmaceutical premix formulation is essentially free of a preservative. In one embodiment, the pharmaceutical premix formulation is stable for at least 6 months at about 25 degrees Celsius.

### DETAILED DESCRIPTION

The present disclosure is generally directed to a pharmaceutical premix formulation comprising from about 0.4 to about 1.1 mg/ml midazolam hydrochloride (HCl), wherein the formulation is an aqueous, premix formulation and has a pH from about 2.5 to about 3.7. A liquid midazolam premix pharmaceutical formulation, as described herein, is ready for administration without the need for dilution to achieve a certain concentration suitable for administration to a human patient. The formulation of the disclosure can be administered to a human patient in need thereof intravenously, e.g., as either a bolus intravenous dose or by continuous intravenous infusion. The midazolam premix pharmaceutical formulation described herein may be aseptically filled into a container, preferably a flexible container fabricated from a multilayer sheeting composed of polypropylene, polyamide, and polyethylene, to form a sterile pharmaceutical midazolam premix product. In certain embodiments, the innermost layer of the flexible container is polyethylene. The midazolam premix pharmaceutical formulation can be a single use premix which is a sterile, stable and ready to use aqueous solution for intravenous (IV) administration, such as IV infusion, including continuous infusion. The midazolam formulation described herein is a ready to use injectable solution.

The disclosed liquid midazolam premix pharmaceutical formulation and containers containing said formulation are advantageous in reducing the risk of contamination and errors associated with dilution, providing time savings, convenience, and reducing waste by eliminating the need for such dilution. The midazolam formulations provided herein are ready to use premix formulations in order to minimize any medication errors and to make administration of midazolam formulations easier for medical professionals for use in an infusion regimen. Further, the premix formulations described herein are stable.

### Definitions

With respect to the terms used in this disclosure, the following definitions are provided. This application will use the following terms as defined below unless the context of the text in which the term appears requires a different meaning.

As used herein, the term “premix” refers to a ready to use, liquid, aqueous solution suitable for direct administration to human patients, including IV infusion, without requiring dilution prior to administration. “Premix” indicates the formulation is already mixed and is suitable for administration to a human patient. Preferably, the premix solution is supplied as a sterile solution, and is stable over its shelf life, as described herein.

The term “pharmaceutical formulation” refers to a preparation which is in such form as to permit the biological activity of an active ingredient, e.g., midazolam HCl, contained therein to be effective, and which contains no additional components which are unacceptably toxic to a subject to which the formulation would be administered.

The term “aqueous” when used in reference to a formulation refers to a liquid formulation in which the solvent is water (e.g., water for injection (WFI)).

As used herein, the term “sterile” is understood to mean free from any bacteria or other living microorganisms.

A “stable” formulation is one in which the active ingredient therein, e.g. midazolam HCl, essentially retains its physical and chemical stability, therefore its biological activity, upon storage.

A “patient”, “subject” or “individual”, used interchangeably herein, is a mammal, preferably a human.

As used herein, an “effective amount” is an amount that provides a nutritional, physiological, or medical benefit to the individual. In one embodiment, an effective amount of midazolam HCl is an amount that provides for induction of general anesthesia.

The term “pharmaceutically acceptable” as used herein refers to substances that do not cause substantial adverse allergic or immunological reactions when administered to a subject.

The terms “substantially no,” “essentially free” or “substantially free” as used in reference to a particular component means that any of the component present constitutes less than about 3.0% by weight, such as less than about 2.0% by weight, less than about 1.0% by weight, preferably less than about 0.5% by weight or, more preferably, less than about 0.1% by weight.

The phrase “consists essentially of” or “consisting essentially of” as used interchangeably herein in reference to a premix formulation, means that the formulation necessarily includes the listed ingredients and is open to unlisted ingredients that do not materially affect the basic nature or stability of the premix formulation, e.g., liquid, sterile, or the activity of the active ingredient, i.e., midazolam HCl.

As used herein, the term “about” means+/−10% of any recited value, or in an alternative embodiment, +/−5% of any recited value. As used herein, this term modifies any recited value, range of values, or endpoints of one or more ranges.

As used herein, the term “injectable pharmaceutical formulation” refers to a composition suitable for administration to a human patient or subject that is essentially free of visible particulates, for example, a composition meeting the requirements of United States Pharmacopeia 33. Chapter. Injections.

### Midazolam Premix Pharmaceutical Formulations and Uses Thereof

A pharmaceutical premix formulation disclosed herein provides multiple advantages over the art. Midazolam hydrochloride (HCl) (also referred to herein as midazolam) is mainly administered by intramuscular or intravenous modes. Generally, midazolam HCl injection is available as a concentrated solution which needs to be diluted to achieve the desired concentration before administration to a human patient as an intravenous bolus or continuous intravenous infusion. The concentrated midazolam formulation, for example, generally needs to be diluted with 0.9% sodium chloride or 5% dextrose in water. Once the diluted solutions are prepared, they are typically not held for more than 4 hours at room temperature or 24 hours under refrigerated conditions.

Disclosed herein are premix aqueous formulations having an amount of midazolam HCl that is suitable for administering to a human patient without first diluting the formulation. A pharmaceutical premix formulation described herein has a midazolam HCl concentration suitable to administer a desired effective dose to a human patient—thus improving efficiency and also minimizing the potential for error that may result from the dilution process. Generally, pharmaceutical premix formulations described herein have a low concentration of midazolam HCl. e.g., less than 5 mg/ml (e.g., 0.4 to 1 mg/ml) which does not require dilution prior to be administered to a patient for sedation.

The pharmaceutical premix formulations described herein include midazolam. Midazolam is a short-acting hypnotic-sedative drug with anxiolytic, muscle relaxant, anticonvulsant, sedative, hypnotic, and amnesic properties. It belongs to a class of drugs called benzodiazepines. Like other benzodiazepines, midazolam binds to benzodiazepine receptors in the brain and spinal cord and is thus used as a short-acting hypnotic-sedative. Midazolam is frequently used in the form of a salt, such as the water-soluble hydrochloride salt (HCl). Chemically, midazolam HCl is 8-chloro-6-(2-fluorophenyl)-1-methyl-4H-imidazo[1,5-a][1,4]benzodiazepine hydrochloride, with a chemical formula C₁₈H₁₃ClFN₃•HCl and a structural formula as follows:

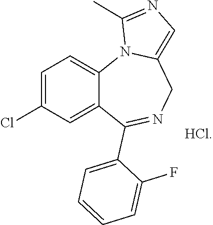 [SMILES: Cc1ncc2n1-c1ccc(Cl)cc1C(c1ccccc1F)=NC2]

Presented herein are premix, aqueous pharmaceutical formulations which are stable and contain midazolam HCl. Notably, the premix pharmaceutical formulations described herein do not need to be diluted prior to administration to a patient. Further, they are stable at room temperature.

Described herein is a pharmaceutical premix formulation comprising from about 0.4 mg/ml to about 1.1 mg/ml midazolam hydrochloride (HCl) and sodium chloride, wherein the formulation is an aqueous, premix formulation and has a pH from about 2.5 to 3.7.

In one embodiment, the pharmaceutical premix formulation disclosed herein comprises midazolam HCl and from about 8 to about 9.5 mg/ml sodium chloride. In other embodiments, the pharmaceutical premix formulation disclosed herein comprises midazolam HCl and about from 8.5 to about 9.5 mg/ml sodium chloride. In other embodiments, the pharmaceutical premix formulation disclosed herein comprises midazolam HCl and from about 8.7 to about 9.1 mg/ml sodium chloride. In one embodiment, the pharmaceutical premix formulation comprises midazolam HCl and about 9.0 mg/ml sodium chloride or about 0.9% sodium chloride.

The amount of midazolam HCl or the amount of salt, e.g., sodium chloride, in the pharmaceutical premix formulation may be described in terms of percentage, by weight per volume (w/v), e.g., 0.9% sodium chloride, or by concentration in the pharmaceutical premix formulation, e.g., 9.0 mg/ml sodium chloride. Concentrations of components of the formulation may also be expressed in terms of molarity (e.g., M or mM); the number of moles or millimoles per liter, respectively).

In one embodiment, a midazolam HCl formulation has a sodium chloride amount from about 0.8% to about 0.945%, from about 0.855% to about 0.945%, about 0.855% to about 0.9%, or about 0.9% to about 0.945%.

In one embodiment, the amount of salt, e.g., sodium chloride, is described in terms of the w/v % of the liquid solution. For example, the pharmaceutical premix formulation may contain from about 0.7% to about 1.1% sodium chloride or about 0.9% sodium chloride.

The osmolality of the pharmaceutical premix formulation comprising midazolam HCl should be suitable for administration. e.g., intravenous, to a human patient. For example, the formulation can have an osmolality from about 270 to about 330 mOsm/kg.

An important feature of the formulation described herein is the concentration of midazolam HCl, as the concentration is such that dilution of the midazolam solution is not required. The stable pharmaceutical premix formulation described herein does not require mixing or diluting prior to delivery to a human patient. The pharmaceutical premix formulation has a midazolam HCl concentration ranging from about 0.05 mg/ml to about 1.1 mg/ml, such as about 0.1, about 0.2, about 0.3, about 0.4, about 0.5, about 0.6, about 0.7, about 0.8, about 0.9, about 1.0, or about 1.1 mg/ml, more preferably from about 0.5 to about 1.0 mg/ml, of midazolam HCl. In one embodiment, a pharmaceutical premix formulation comprises from about 0.4 mg to about 1.1 mg/mi midazolam HCl. In other embodiments, the concentration of midazolam HCl is from about 0.5 mg to about 1.0 mg/ml. In specific embodiments, the concentration of midazolam HCl is about 0.5 mg/ml. In other specific embodiments, the concentration of midazolam HCl is about 1 mg/ml.

As described in more detail below, the pharmaceutical premix formulation may be contained in a flexible bag, such as a non-polyvinyl chloride (NPVC) flexible container. In some embodiments, the pharmaceutical premix formulation of midazolam HCl comprises 25 mg/50 mL in sodium chloride injection in a flexible container made from non-PVC container, e.g., a container fabricated from a multilayer sheeting composed of polypropylene (PP), polyamide (PA) and polyethylene (PE) (VIAFLO), or alternatively, a non-polyvinyl chloride (NPVC) flexible container. In other embodiments, the pharmaceutical premix formulation of midazolam comprises about 50 mg/100 mL in sodium chloride injection in a flexible container. In some embodiments, the pharmaceutical premix formulation of midazolam HCl comprises about 50 mg/50 mL in sodium chloride injection in a flexible container. In some other embodiments, the pharmaceutical premix formulation of midazolam HCl comprises about 100 mg/100 mL in sodium chloride injection in a flexible container.

The total mg amount of midazolam HCl in a formulation described herein can range in part based on the volume of the flexible container containing the formulation, e.g., about 25 mg, about 50 mg, or about 100 mg per bag. For example, each ready to use bag containing the pharmaceutical premix formulation disclosed herein could represent approximately a day's supply of medication which is appropriate for an ICU sedation indication. In some embodiments, the pharmaceutical premix formulation provided herein comprises about 50 mg of midazolam HCl. In some other embodiments, the pharmaceutical premix formulation provided herein comprises about 100 mg of midazolam. HCl. The dose is variable based on patient weight and patient response.

The pH of the pharmaceutical premix formulation described herein is a pH that maintains the stability of midazolam HCl. The pH of the pharmaceutical premix formulation is in the range from about 2.2 to about 4.0, such as, for example, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0. In certain embodiments, the pharmaceutical premix formulation has a pH from about 2.5 to 3.7. In other embodiments, pharmaceutical premix formulation has a pH from about 2.8 to 3.4. In some embodiments, the formulation has a pH in a range from about 2.5 to about 2.99, such as from about 2.50, about 2.55, about 2.60, about 2.65, about 2.70, about 2.75, about 2.80, about 2.85, about 2.90, or about 2.95. In some embodiments, the formulation has a pH in a range from about 2.5 to about 2.95. In some embodiments, the formulation has a pH of 3.0.

The pH of the solution may be adjusted by use of a pH adjusting agent, and optionally, if needed, a buffer may be used to maintain the pH in the said range. The pH adjusting agent that may be used includes, but is not limited to sodium hydroxide, potassium hydroxide, hydrochloric acid, sulphuric acid, acetic acid, sodium acetate, tartaric acid, and the like, and mixtures thereof. In one embodiment, the pH adjusting agent is sodium hydroxide, hydrochloric acid, or a combination thereof. Thus, in certain embodiments, the pharmaceutical premix formulations provided herein comprise hydrochloric acid (e.g., about 0.2 to about 0.5 mg/ml, such as about 0.22 mg/ml, or about 0.44 mg/ml) and sodium hydroxide.

In some embodiments, each mL of the pharmaceutical premix formulation provided herein contains: about 0.5 mg midazolam HCl, water for injection q.s., about 9 mg/ml sodium chloride, about 0.22 mg/ml hydrochloric acid, sodium hydroxide and, if necessary, sufficient hydrochloric acid to provide a pH from about 2.5 to about 3.7.

In some other embodiments, each mL of the pharmaceutical premix formulation provided herein contains: about 1 mg midazolam HCl, water for Injection q.s., about 9 mg/ml sodium chloride, about 0.44 mg/ml hydrochloric acid, sodium hydroxide and, if necessary, sufficient hydrochloric acid to provide a pH from about 2.5 to about 3.7.

In some embodiments, the pharmaceutical premix formulation provided herein comprises midazolam HCl at a concentration from about 0.5 mg/ml to about 1.0 mg/ml, and from about 8.5 mg/ml to about 9.5 mg/ml sodium chloride, and having a pH from about 2.2 to about 4.0. In one embodiment, the pharmaceutical premix formulation provided herein comprises midazolam HCl at a concentration of about 0.5 mg/ml, and 9.0 mg/ml sodium chloride, has a pH from about 2.5 to 3.7, such as a pH of about 3.0.

In one other embodiment, the pharmaceutical premix formulation provided herein comprises midazolam HCl at a concentration of about 1.0 mg/ml, and 9.0 mg/mi sodium chloride, has a pH from about 2.5 to about 3.7, such as a pH of 3.0. In one embodiment, the pharmaceutical premix formulation provided herein comprises midazolam HCl at a concentration of about 0.5 mg/ml, and about 0.9% sodium chloride, and has a pH from about 2.5 to about 3.7.

In one other embodiment, the pharmaceutical premix formulation provided herein comprises midazolam HCl at a concentration of about 1.0 mg/ml, and about 0.9% sodium chloride, and has a pH from about 2.5 to about 3.7.

In any of the foregoing embodiments, the pharmaceutical premix formulation may have a pH from about 2.50 to about 2.95.

The pharmaceutical premix formulation of the disclosure may contain one or more impurities, Such impurities may be degradants (i.e., impurities formed via degradation of one or more components of the composition over time, such as upon storage), impurities initially present in the midazolam HCl (i.e., impurities formed during the synthesis of midazolam HCl), or both. Degradants may be formed via degradation of one or more components of the formulation over time. Sources of degradation include, but are not limited to, oxidation, racemization, visible light, ultraviolet light, moisture, heat, changes in pH, and composition component interactions. Impurities such as degradants can be as measured using techniques known in the art, such as HPLC/UV, ultra-performance liquid chromatography (UPLC), gas chromatography-mass spectrometry (GCMS), liquid chromatography-mass spectrometry (LCMS), or inductively coupled plasma mass spectroscopy (ICPMS). The pharmaceutical premix formulation of the disclosure may be characterized according to stability. Stability of a pharmaceutical premix formulation can be determined by assaying the level of impurities (e.g., degradant(s)) over a period of time. Preferably, minimal or non-detectable levels of degradant are found over a testing period.

In some embodiments, the pharmaceutical premix formulation comprises the isomeric impurity midazolam 6H-isomer. The 6H isomer is a degradant of the active compound, and may also be referred to as Midazolam EP impurity B, 8-Chloro-6-(2-fluorophenyl)-1-methyl-6H-imidazo[1,5-a][1.4]benzodiazepine, 8-chloro-6-(2-fluorophenyl)-1-methyl-3a,6-dihydro-3H-benzo[f]imidazo[1,5-a][1,4]diazepine, or isomidazolam. Midazolam 6H-isomer (CAS #59469-74-8) has the structural formula:

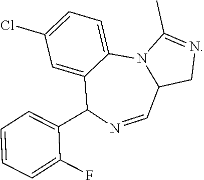 [SMILES: CC1=NCC2C=NC(c3ccccc3F)c3cc(Cl)ccc3N12]

In some embodiments, the pharmaceutical premix formulation comprises the degradant impurity reduced reduced midazolam. The reduced reduced midazolam is a degradant of the active compound. This impurity may also be referred to as Midazolam EP Impurity J, or 8-chloro-6-(2-fluorophenyl)-1-methyl-3a,4,5,6-tetrahydro-3H-imidazo[1,5-a][1.4]benzodiazepine. Reduced reduced midazolam (CAS #59469-08-8) has the structural formula:

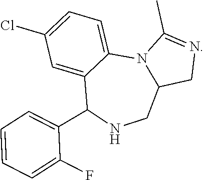 [SMILES: CC1=NCC2CNC(c3ccccc3F)c3cc(Cl)ccc3N12]

The pharmaceutical premix formulation of the disclosure may be characterized as having a low or non-detectable level of impurity, such as midazolam 6H-isomer and/or reduced reduced midazolam. Levels of these impurities relevant to the stable formulations disclosed herein are provided in the tables in the examples.

For example, a midazolam premix formulation may contain no more than about 0.5% midazolam 6H-isomer. In one embodiment, a midazolam premix formulation contains no more than about 0.4% midazolam 6K-isomer, no more than about 0.3% midazolam 6H-isomer, no more than about 0.2% midazolam 6H-isomer, no more than about 0.1% midazolam 6H-isomer, no more than about 0.05% midazolam 6H-isomer, or no more than about 0.01% midazolam 6H-isomer.

In another example, a midazolam premix formulation may contain no more than about 0.5% reduced reduced midazolam. In one embodiment, a midazolam premix formulation contains no more than about 0.4% reduced reduced midazolam, no more than about 0.3% reduced reduced midazolam, no more than about 0.2% reduced reduced midazolam, no more than about 0.1% reduced reduced midazolam, no more than about 0.05% reduced reduced midazolam, or no more than about 0.01% reduced reduced midazolam.

The pharmaceutical premix formulation of the disclosure may be characterized according to stability, such as long-term stability to storage. For example, in some embodiments, the pharmaceutical premix formulation is stable for at least about 3 months or at least about 6 months of storage at about 25 degrees Celsius. For example, in some embodiments, the pharmaceutical premix formulation is stable for at least 3 months of storage at about 40 degrees Celsius.

In one embodiment, a pharmaceutical premix formulation comprises midazolam HCl and comprises a low level of midazolam related impurities. In some embodiments, the pharmaceutical premix formulation contains less than about 1%, less than about 0.5%, or less than about 0.2% of impurities. In one embodiment, a pharmaceutical premix formulation comprising midazolam HCl contains a pharmaceutically acceptable level of midazolam related impurity(ies). In some embodiments, midazolam related impurities are midazolam 6H-isomer, reduced reduced midazolam, or a combination thereof.

Stability of the pharmaceutical premix formulation is achieved without the use of a preservative. Thus, in certain embodiments, a pharmaceutical premix formulation of the disclosure comprising midazolam HCl is essentially free of a preservative. Examples of preservatives include, but are not limited to, sodium benzoate, EDTA, sorbic acid, and parabens.

In certain embodiments, the pharmaceutical premix formulation comprises from about 0.5 to about 5 mg/ml midazolam HCl, and from about 8.4 to about 9.4 mg/ml sodium chloride. In one embodiment the formulation is an aqueous, premix formulation and has a pH from about 2.5 to 3.7. Further, the formulation is essentially free of a preservative.

In certain embodiments, the pharmaceutical premix formulation consists essentially of about 0.4 to about 1.1 mg/ml midazolam HCl, and from about 8.5 to about 9.5 mg/ml sodium chloride, wherein the formulation is an aqueous, premix formulation and has a pH from about 2.5 to 3.7.

In certain embodiments, provided herein is a room temperature stable, ready to use premix formulation of midazolam HCl at a concentration of 0.5 mg/mL in 0.9% sodium chloride (in a flexible container such as a 50 or 100 mL VIAFLO Container Closure System) in diluted form, which can be used for as a continuous infusion depending on dosage requirement. In certain other embodiments, provided herein is a room temperature stable, ready to use premix formulation of midazolam HCl at a concentration of 1 mg/mL in 0.9% sodium chloride (in a flexible container such as a 50 or 100 mL VIAFLO Container Closure System) in diluted form, which can be used for as a continuous infusion depending on dosage requirement. In a preferred embodiment, the pharmaceutical premix formulation is not diluted prior to intravenous infusion into a subject (e.g., human patient).

The midazolam HCl premix formulation of the disclosure may be packaged in any suitable primary container known in the art including but not limited to vials, syringes, bags, bottles and ampules presentations. Containers may be fabricated from glass or from polymeric materials. Suitable containers, such as primary containers, described further herein below, include flexible containers such as flexible bags. Primary is used to describe the container which directly houses the premix formulation.

In a preferred embodiment, a midazolam premix formulation described herein can be contained in a flexible container. In some embodiments, the flexible container described herein consist essentially of the midazolam pharmaceutical premix formulation provided herein.

A flexible container, such as a bag, provides better control relative to a rigid container, providing enhanced safety. Flexible bags suitable as containers include those disclosed in US 2008/0249499, which is hereby incorporated by reference in its entirety. Other flexible bags may be used. Preferred flexible bag primary containers may be free of PVC (non-PVC), such as those disclosed in U.S. Pat. Nos. 5,849,843 and 5,998,019, which are hereby incorporated by reference in their entirety. Suitable flexible polymeric primary containers include but are not limited to GALAXY IV containers (Baxter International Inc.), VIAFLO containers (Baxter International Inc.), and INTRAVIA containers (Baxter International Inc.). In one embodiment, a flexible container used to house the midazolam premix formulation is a flexible plastic container fabricated from a multilayer sheeting composed of polypropylene (PP), polyamide (PA) and polyethylene (PE).

Preferably, the flexible container containing a midazolam premix formulation described herein is a ready to use intravenous bag.

The volume capacity of the flexible container can range, for example, from about 50 mL to about 1000 mL. In certain embodiments, the volume capacity of each flexible container may range from about 50 ml to about 500 ml, such as for example about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440 or about 450 ml, more preferably from about 50 ml to about 100 ml. According to some embodiments, the container (e.g., infusion bag) can accommodate a volume from about 100 ml to about 200 ml, preferably about 100 ml. In certain embodiments, the volume of the flexible container is about 50 mL. In certain embodiments, the volume of the flexible container is about 100 mL. In certain embodiments, the volume of the flexible container is about 250 mL. In certain embodiments, the volume of the flexible container is about 500 mL. In certain embodiments, the volume of the flexible container is about 1000 mL. The flexible container, e.g., a plastic bag, may be suitable for intravenous infusion of the formulation to a human subject. In some embodiments, the ready-to-use, sterile, stable premix formulation of midazolam filled in the flexible container comprises a concentration in the range of about 0.5 mg/ml to about 1.0 mg/ml of midazolam HCl. In one embodiment, the pharmaceutical premix formulation of midazolam HCl filled in the flexible container comprises a concentration of about 1.0 mg/ml of midazolam HCl. In another embodiment, the pharmaceutical premix formulation of midazolam HCl filled in the flexible container comprises a concentration of about 0.5 mg/ml of midazolam HCl.

The material of the flexible container can be a plastic, e.g., the container may include polypropylene (PP), polyamide (PA), and polyethylene (PE) (which are the plastics that make up the VIAFLO flexible container). In certain embodiments, the flexible container is a VIAFLOW container, which is a bag composed of polyolefin/polyamide co-extruded plastic. The premix formulation may be administered to a human patient intravenously via an infusion tube connected to the flexible container. A flexible container also provides better control over a rigid container for safety purposes. By avoiding dilution, where the sterile premix formulation is ready to use, contamination and/or medication error can be avoided. In some embodiments, the flexible container is part of an intravenous hook system such that the intravenous hook comprises the flexible container with the stable, ready-to-use pharmaceutical premix formulation of midazolam HCl. In some embodiments, the flexible container is a flexible bag which is suitable for intravenous use, i.e, an IV bag. Such flexible bags may be formed by any of a number of methods, for example, by an exemplary form/fill/seal process where a sheet layer (or film) is aligned and then folded by a folding triangle. After that aligning and folding step, the film can be cut to allow the introduction of a port system between the two resulting facing films. The port system can then be automatically fed in place and welded between the two opposing faces of the folded film. By vertical welding, the bottom part of the bag is formed and a hanger hole is punched. The side of the bag is formed by horizontal welding, while the solution for infusion is fed into the formed flexible bag. The upper horizontal welding also can form the lower side of the next bag. Finally, each bag is separated from the other during a sealing/cutting process. Thus, the flexible bag may be formed of a single sheet layer of flexible material, folded and sealed along the peripheral edges. Examples of suitable flexible containers are described in US 2008/0249499 and US 2021/0275470, each of which is hereby incorporated by reference in their entirety with respect to the flexible containers described therein.

In other methods, two flexible sheets are joined at a top end and two side edges, i.e., when the two flexible sheets are placed in facing relationship they can be joined at their overlying/overlapping peripheral edges, while leaving an opening at a bottom end. The sealed top end and side edges, along with the open bottom end, are collectively referred to herein as the peripheral edges of the flexible bag. The top end includes a hanger aperture, which is preferably laterally offset from a central vertical axis of the flexible bag portion. The port system can then be fed in place and welded between the two opposing flexible sheets. Any other known method of bag manufacture, such as blow molding or vacuum forming, may also be used.

In one embodiment, a multilayer flexible sheet layer (or film) is used to manufacture the flexible container. In one aspect according to this embodiment, the multilayer flexible sheet layer comprises a low-density polyethylene (PE) bottom (inside facing when assembled) layer such as Stamylex 1026F, a polypropylene (PP) top (outside facing when assembled) layer such as Bormed RD804CF or Bormed RE816CF, and a polyamide (PA) middle layer such as Grilon FG40NL Natural 6021. A composite PE bottom layer may also be used. A composite PP top layer may also be used. Adhesion between the PP and PA layers may be achieved by a tie layer comprising PP grafted with maleic anhydride such as Admer QF300E. Similarly, adhesion between the PA and PE layers may be achieved by a tie layer comprising PE grafted with maleic anhydride such as Yparex 8104E. In some embodiments, the flexible sheet does not comprise any cycloolefin polymers or cycloolefin copolymers.

The flexible sheet layer may have any suitable thickness, for example, between about 100 pm and about 250, pm, between about 125 pm and about 225, pm, and/or between about 150 pm and about 200, pm.

Such flexible bags necessarily include an administration port. In one embodiment, the primary container is a flexible bag with a single port adapted for fluid delivery (internally or externally), the single port comprising an administration port for delivering formulations comprising an oxygen-sensitive pharmaceutical compound, for example, the ready-to-use norepinephrine formulations disclosed herein, to a patient via connection to an administration set. Because the primary container includes a ready-to-use formulation therein, the primary container according to this embodiment does not include a separate functioning “injection” or “reconstitution” port that allows a fluid to be added to the formulation of the primary container. Instead, the primary container may include a non-accessible “dummy” port that does not include a frangible cannula or pierceable diaphragm or membrane and thus does not readily allow for communication between an interior of the primary container and the exterior. A “dummy” port is not considered to be a port as conventionally understood as it does not function provide access to the contents of the primary container. The dummy port may be substantially solid. As a result, the dummy port may be configured to prevent at least 90% of attempts to insert a standard 21 gauge needle at an insertion rate of 200 mm/min into the interior of the primary container using an insertion force of 5 N or less, or even 10 N or less.

The port structure is preferably sized and configured to fit within the opening (not illustrated) in the bottom end of the bag. In a preferred embodiment, the opening and port structure are substantially as wide as the top edge, such that the port structure essentially defines the entire bottom surface of the bag. The port structure is formed of a plastic material that in the preferred embodiment is less flexible than the bag portion and is preferably molded as a single, integral unit, in this respect, the preferred port structure may be considered as having intermediate rigidity, as it is preferably more rigid than the generally flexible bag, but less than completely rigid such as glass or metal. The port structure typically includes a single functioning port as mentioned above.

In one embodiment, the port structure is molded from PE. The molded port system includes a non-accessible (dummy) port, which prevents addition of medication or diluent, and an administration port, which allows access to the bag contents as described above. The administration port may have a twist-off protective cap and a membrane in addition to a flexible sleeve. The twist off protector and the membrane may comprise PE. The flexible sleeve typically provides a non-dripless access connector (non-DAC) port configuration and in one embodiment may comprise a coextruded inner layer and outer layer. In one aspect, the inner layer may comprise a blend of polyurethane and silicone, such as TPSiV 3111-70A, and the outer layer may comprise an ethylene-vinyl acetate copolymer such as Ateva 1807 EG. The port system may be gamma irradiated prior to incorporation into the flexible bag structure.

In one embodiment, the midazolam HCl premix pharmaceutical formulation is contained in a packaged, sealed container system. In some embodiments, the packaged, sealed container system comprises a primary container (as disclosed herein and referred to as a flexible container) including a ready-to-use pharmaceutical premix formulation of midazolam HCl therein, and further includes a secondary container. The primary container may be disposed within and enclosed by the secondary container. The secondary container may be an overpouch container. Overpouches are flexible containers that can be used as secondary containers in the packaged, sealed container system to store, protect, and transport the primary container containing the midazolam premix pharmaceutical formulation. Generally, overpouches are provided by a first flexible sheet layer, an opposing second flexible sheet layer, and a seal disposed along a common peripheral edge of the first and second flexible sheet layers. Preferably, the secondary overpouch container should be optically transparent to enable visual inspection of the primary container and any other contents within the overpouch. It is also desirable for the overpouch container to be capable of withstanding autoclaving or other terminal sterilization process without causing the primary container therein to shrink/wrinkle and without becoming discolored and/or adhered to the primary container

The overpouch container may be an aluminum overpouch, a light absorbing polymeric overpouch, or a similar barrier structure. In one embodiment, the primary container is in fluid communication with any other contents of the overpouch secondary container.

In one embodiment, the overpouch secondary container comprises a first flexible sheet layer comprising an amber transparent film and a second flexible sheet layer comprising an opaque aluminum laminated foil. The first flexible sheet layer may be an amber transparent multilayer film comprising a PET (Polyethylene terephthalate)/PA/PP laminate to allow the contents within the secondary container, for example, any labeling on the primary container to be seen. The second flexible sheet layer may be an opaque laminated foil comprising a PET/PA/Aluminum foil/PP laminate. A seal is disposed along a common peripheral edge of the first and second flexible sheets.

In another embodiment, the overpouch secondary container comprises a first flexible single layer sheet layer comprising a polymeric blend of a high density polyethylene and a surface enhancing polymer, an opposing second flexible single layer sheet layer comprising a polymeric blend of a high density polyethylene and a surface enhancing polymer of an ethylene propylene diene terpolymer dispersed in a polyolefin matrix, and a seal disposed along a common peripheral edge of the first and second flexible sheets as disclosed in US 2006/0240204, which is hereby incorporated by reference in its entirety.

An oxygen scavenger may be disposed within and enclosed by the overpouch secondary container. A sachet located adjacent to the primary container and disposed within the secondary overpouch container may include the oxygen scavenger. The sachet (i.e., a bag) itself is porous and may comprise polyethylene materials. The oxygen scavenger may comprise iron powder, iron oxide powder, or a mixture thereof, for example, micronized iron. Other known oxygen scavengers may also be used. The oxygen scavenger is primarily included to absorb small amounts of oxygen that permeate through the secondary container during the shelf life of the drug product.

Thus, despite the primary and secondary containers being sealed, the fluid contents of the primary container may be considered to be in fluid communication with the contents of the secondary container, including the oxygen scavenger. Thus, the oxygen scavenger can be considered to be in fluid communication with the formulation of the primary container.

The premix formulation may be administered to a human patient intravenously via an infusion tube connected to the flexible container. In some embodiments, the flexible container is part of an intravenous hook system such that the intravenous hook comprises the flexible container with the stable, ready-to-use pharmaceutical premix formulation of midazolam HCL.

Generally, midazolam injection is available as a concentrated solution which needs to be diluted before administration as an intravenous bolus or continuous intravenous infusion to achieve the desired concentration. The concentrated midazolam formulation, for example, generally needs to be diluted with 0.9% sodium chloride or 5% dextrose in water. A typical initial dose of midazolam is 40 mg injected slowly intravenously (over 1 to 2 minutes). If a satisfactory response does not occur within 1 hour, the dose may be increased to 80 mg injected slowly intravenously (over 1 to 2 minutes).

The pharmaceutical premix formulations and the flexible container disclosed herein provide advantages over those other midazolam formulations known in the art. For example, a premix liquid formulation, like those disclosed herein, provides a certain dose amount (e.g., an IV bolus/Infusion) that is readily available to the patient without a need to first dilute the midazolam formulation. By providing a pharmaceutical premix formulation which is ready to use (i.e., which does not require dilution), the risk of contamination and compounding, or medication error associated therewith, is essentially eliminated. Furthermore, the flexible container of the present disclosure provides a means of direct intravenous administration of the sterile, stable formulation of midazolam HCl to the patient through the infusion container, using the outlet port. Further, the formulation disclosed herein reduces time needed by medical experts to dilute and prepare midazolam HCl for administration, and also reduces medical waste.

Importantly, as noted above, the premix formulation described herein, which can be contained in a flexible container such as a plastic bag suitable for intravenous infusion, are ready to use for delivery of the midazolam to the patient, i.e., there is no need to dilute the midazolam and the pharmaceutical premix formulation contained within the flexible container can be administered to the patient without a mixing and/or dilution step. In one embodiment, the system described herein (i.e., pharmaceutical premix formulation in a flexible container, such as a VIAFLO plastic bag), provides improved safety for the patient by providing better control in contrast to rigid containers used to mix and dilute midazolam to achieve a suitable concentration for administering an accurate dose, e.g., 40 mg, of midazolam HCl to a patient.

In some aspects, there are provided methods of using the stable, ready to use pharmaceutical premix formulation of midazolam HCl. Disclosed herein are methods of sedating a human subject in need thereof. In certain embodiments, the pharmaceutical premix formulation of midazolam HCl is administered preoperatively to potentiate the effect of an anesthetic, wherein administration of the formulation reduces the amount of anesthetic required to achieve a desired level of anesthesia. In certain embodiments, the premix formulation of midazolam can be administered as an anxiolytic analgesic premedication prior to the operation with or without administration of an amount of an anesthetic effective to achieve a desired level of local or general anesthesia.

In one embodiment, a method of sedating a human subject in need thereof is described herein. The method comprises the steps of obtaining the flexible container provided herein comprising the pharmaceutical premix formulation described above, placing the flexible container on a hook or means for suspending the flexible container, and intravenously administering the liquid formulation into the human subject.

In another embodiment, a method of sedating a human subject in need thereof is described herein. The method comprises intravenously administering to the human subject a pharmaceutical premix formulation comprising from about 0.5 mg/ml to about 1.1 mg/ml, such as for example about 0.5, about 0.6, about 0.7, about 0.8, about 0.9, about 1.0, or about 1.1 mg/ml, more preferably from about 0.5 to 1.0 mg/ml, of midazolam HCl. In some embodiments, the liquid formulation in the method described above further comprises about 0.8% to about 0.9% sodium chloride. In one embodiment, the method comprises intravenously administering a pharmaceutical premix formulation comprising about 0.5 mg/ml midazolam HCl and 0.9% sodium chloride, to the human subject. In another embodiment, the method comprises intravenously administering a pharmaceutical premix formulation comprising about 1.0 mg/ml midazolam HCl and about 0.9% sodium chloride, to the human subject. In one embodiment, the method comprises intravenously administering a pharmaceutical premix formulation comprising 50 mg midazolam HCl at a concentration about 0.5 mg/ml midazolam HCl and 0.9% sodium chloride, to the human subject. In another embodiment, the method comprises intravenously administering a pharmaceutical premix formulation comprising about 100 mg midazolam HCl to a concentration of about 1.0 mg/ml midazolam HCl and about 0.9% sodium chloride, to the human subject. In the embodiments of the methods described above, the formulation is an aqueous, premix formulation that is not diluted prior to administration to the human subject.

In one embodiment, the pharmaceutical premix formulation comprising midazolam HCl is stored and infused from a flexible container such as a 50 or 100 mL VIAFLO Container Closure System, which can be used for continuous infusion depending on dosage requirement. In some embodiments, the VIAFLO Container is part of an intravenous hook system such that the intravenous hook comprises the VIAFLO Container with the pharmaceutical premix formulation of midazolam HCl.

Pharmaceutical premix formulations described herein comprising midazolam HCl may be essentially free of a preservative.

In certain embodiments, midazolam HCl is administered as a continuous intravenous dose to a human subject for sedation at a initial infusion rate of between about 1 mg/hr and about 7 mg/hr, or between about 1 mg/hr and about 1.5 mg/hr, or between about 1 mg/hr and about 2 mg/hr, or between about 1 mg/hr and about 2.5 mg/hr, or between about 1 mg/hr and about 3 mg/hr, or between about 1 mg/hr and about 3.5 mg/hr, or between about 1 mg/hr and about 4 mg/hr. or between about 1 mg/hr and about 4.5 mg/hr, or between about 1 mg/hr and about 5 mg/hr, or between about 1 mg/hr and about 6 mg/hr.

In some embodiments, midazolam HCl is administered as a continuous intravenous dose for a period of time of between about 1 and about 10 minutes, or between about 1 and about 20 minutes, or between about 1 and about 30 minutes, or between about 1 and about 2 hours, or between about 1 and about 3 hours, or between about 1 and about 4 hours, or between about 1 and about 5 hours, or between about 1 and about 6 hours, or between about 1 and about 7 hours, or between about 1 and about 8 hours, or between about 1 and about 9 hours, or between about 1 and about 10 hours, or between about 1 and about 11 hours, or between about 1 and about 12 hours, or between about 1 and about 13 hours, or between about 1 and about 14 hours, or between about 1 and about 15 hours, or between about 1 and about 16 hours, or between about 1 and about 17 hours, or between about 1 and about 18 hours, or between about 1 and about 19 hours, or between about 1 and about 20 hours, or between about 1 and about 21 hours, or between about 1 and about 22 hours, or between about 1 and about 23 hours, or between about 1 and about 24 hours.

In certain embodiments, the pharmaceutical premix formulation disclosed herein be administered to a human subject as a perioperative treatment. In certain embodiments, the formulation can be administered as a premedication prior to an operation. In certain embodiments, the pharmaceutical premix formulation disclosed herein can be used in the manufacture of a medicament for perioperative treatment of a human subject to reduce the responses of the autonomic nervous system to stressful stimuli during an operation. In some embodiments, the pharmaceutical premix formulation disclosed herein be administered to a human subject as an adjunct anesthesia. For example, the formulation can be administered with or without an amount of an anesthetic effective to achieve a desired level of local or general anesthesia. In certain embodiments, administration of the pharmaceutical premix formulation disclosed herein reduces the amount of anesthetic required to achieve a desired level of anesthesia. Since midazolam induces only sedation, management of analgesia by administration of an analgesic agent may be required to control the pain during and post-operative surgery.

In certain embodiments, the pharmaceutical premix formulation disclosed herein do not include any other active ingredient, or therapeutic agent, other than midazolam HCl.

The total mg of midazolam HCl in the pharmaceutical premix formulation described herein (also described in terms of the amount of pharmaceutical premix formulation in a flexible container) can be, for example 10-120 mg. In one embodiment, a flexible container is a ready to use bag, e.g., a container closure system such as VIAFLO, and contains 100 mg of midazolam HCl in 100 ml (i.e., 100 mg/100 mL concentration in a premix formulation), so the flexible container comprises approximately a day's supply of medication which is appropriate for an ICU sedation indication. In another embodiment, a flexible container is a ready to use bag, e.g., a container closure system such as VIAFLO, and contains 50 mg of midazolam HCl in 100 ml (i.e., 50 mg/100 mL concentration in a pharmaceutical premix formulation).

All methods described herein can be performed in any suitable order unless otherwise indicated herein or otherwise clearly contradicted by context. The use of any and all examples, or exemplary language (e.g., “such as”) provided herein, is intended merely to better illustrate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents.

Reference throughout this specification to “one embodiment.” “certain embodiments,” “one or more embodiments” or “an embodiment” means that a particular feature, structure, material, or characteristic described in connection with the embodiment is included in at least one embodiment of the technology. Thus, the appearances of phrases such as “in one or more embodiments,” “in certain embodiments,” “in one embodiment” or “in an embodiment” in various places throughout this specification are not necessarily referring to the same embodiment of the technology. Furthermore, the particular features, structures, materials, or characteristics may be combined in any suitable manner in one or more embodiments. Any ranges cited herein are inclusive.

Various changes and modifications to the presently preferred embodiments disclosed herein will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1: Stability of Midazolam Premix Formulation in VIAFLO

Formulations described in Tables 1 to 4 contained 0.5 mg/ml or 1 mg/ml midazolam HCl and 0.9% sodium chloride, and had a pH of 2.5 to 3.5. The formulations were tested in a 50 mL or 100 mL VIAFLO pack. The results show that the premix formulations were stable in the VIAFLO containers. Impurities reduced reduced midazolam (8-chloro-6-(2-fluorophenyl)-1-methyl-3a,4,5,6-tetrahydro-3H-imidazo[1,5-a][1,4]benzodiazepine) and 6H-midazolam isomer (8-Chloro-6-(2-fluorophenyl)-1-methyl-6H-imidazo[1,5-a][1,4]benzodiazepine) were quantified using HPLC (throughout examples).

TABLE 1
1 mg/ml in 100 mL VIAFLO pack

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 Months | 12 Months |
| 1. | Description | A clear, colorless | * | * | * |
| | | solution filled in viaflo | | | |
| | | bag | | | |
| 2. | Clarity of | Clear solution | # | # | # |
| | Solution | essentially free from | | | |
| | | visible particulate matter | | | |
| 3. | Color and | Color of solution should | @ | @ | @ |
| | achromicity of | not be more intense than | | | |
| | solution | the USP matching fluid | | | |
| | | A | | | |
| 4. | pH | 2.5 to 3.5 | 3.39 | 3.42 | 3.41 |
| 5. | Assay of Midazolam | 90.0% to 110.0% | 95.12 | 100.07 | 100.80 |
| 6. | Organic impurity | | | | |
| | Reduced reduced | NMT 0.5% | ND | ND | ND |
| | midazolam | | | | |
| | 6H-midazolam | NMT 0.5% | ND | 0.003 | 0.004 |
| | isomer | | | | |
| | Any unspecified | NMT 0.1% | 0.008 | 0.018 | 0.017 |
| | Impurity | | | | |
| | Total Impurities | NMT 1.1% | 0.083 | 0.108 | 0.136 |
| 7 | Particulate matter | For >10 μm: -Not | 10 μm: 123.3 | 10 μm: 1686.7 | 10 μm: 126.7 |
| | | More Than 6000 | | | |
| | | particles per container | | | |
| | | For >25 μm: -Not | 25 μm: 0.0 | 25 μm: 166.7 | 25 μm: 20.0 |
| | | More Than 600 | | | |
| | | particles per container | | | |
| 8 | Osmolality | Between 270 mOsm/kg | 290 | NA | NA |
| | | and 330 mOsm/kg | mOsm/kg | | |

* Clear colorless solution
# Clear solution essentially free from visible particulate matter
@ Color of solution is less intense than the USP matching fluid A
ND: Not detected
NMT throughout is “not more than”

TABLE 2
1 mg/ml in 50 mL VIAFLO pack

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 Months | 12 Months |
| 1. | Description | A clear, colorless | * | * | * |
| | | solution filled in viaflo | | | |
| | | bag | | | |
| 2. | Clarity of | Clear-solution | # | # | # |
| | Solution | essentially free from | | | |
| | | visible particulate | | | |
| | | matter | | | |
| 3. | Color and | Color of solution should | @ | @ | @ |
| | achromicity of | not be more intense than | | | |
| | solution | the USP matching fluid | | | |
| | | A | | | |
| 4. | pH | 2.5 to 3.5 | 3.36 | 3.34 | 3.42 |
| 5. | Assay of Midazolam | 90.0% to 110.0% | 96.91 | 101.29 | 100.7 |
| 6. | Organic impurity | | | | |
| | Reduced reduced | NMT 0.5% | ND | ND | ND |
| | midazolam | | | | |
| | 6H-midazolam | NMT 0.5% | ND | 0.003 | 0.004 |
| | isomer | | | | |
| | Any unspecified | NMT 0.1% | 0.010 | 0.024 | 0.017 |
| | Impurity | | | | |
| | Total Impurities | NMT 1.1% | 0.098 | 0.133 | 0.141 |
| 7 | Particulate matter | For >10 μm: -Not More | 10 μm: 90.0 | 10 μm: 450 | 10 μm: 90.0 |
| | | Than 6000 particles per | | | |
| | | container | | | |
| | | For >25 μm: -Not | 25 μm: 00.0 | 25 μm: 16.7 | 25 μm: 00.0 |
| | | More Than 600 | | | |
| | | particles per container | | | |
| 8 | Osmolality | Between 270 mOsm/kg | 290 | NA | NA |
| | | and 330 mOsm/kg | mOsm/kg | | |

* Clear colorless solution
# Clear solution essentially free from visible particulate matter
@ Color of solution is less intense than the USP matching fluid A
ND: Not detected

TABLE 3
0.5 mg/ml in 100 mL VIAFLO pack

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 Months | 6 Months |
| 1. | Description | A clear, colorless | * | * | * |
| | | solution filled in | | | |
| | | viaflo bag | | | |
| 2 | Clarity of | Clearsolution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | ntense than the USP | | | |
| | | Hatching fluid A | | | |
| 4. | pH | 2.5 to 3.5 | 3.55 | 3.44 | 3.34 |
| 5. | Assay of Midazolam | 90.0% to 110.0% | 97.70 | 99.73 | 99.16 |
| 6. | Organic impurity | | | | |
| | Reduced reduced | NMT 0.5% | ND | ND | ND |
| | midazolam | | | | |
| | 6H-midazolam | NMT 0.5% | 0.005 | 0.008 | 0.008 |
| | Any unspecified | NMT 0.1% | 0.010 | 0.023 | 0.015 |
| | Impurity | | | | |
| | Total Impurities | NMT 1.1% | 0.106 | 0.155 | 0.145 |
| 7 | Osmolality | Between 270 | 290 mOsm/kg | NA | NA |
| | | mOsm/kg and 330 | | | |
| | | mOsm/kg | | | |

* Clear colorless solution
# Clear solution essentially free from visible particulate matter
@ Color of solution is less intense than the USP matching fluid A
ND: Not detected

TABLE 4
0.5 mg/ml in 50 mL VIAFLO pack

| | | | | 40° C. ± 2° C./ | |
|---|---|---|---|---|---|
| | | | | NMT | 25° C. ± 2° C./ |
| | | | | 25% RH | 40% ± 5% RH |
| Sr. | | | | (Horizontal) | (Horizontal) |
| No. | Test Parameters | Limit | Initial | 6 Months | 12 Months |
| 1. | Description | A clear, colorless | * | * | * |
| | | solution filled in | | | |
| | | viaflo bag | | | |
| 2. | Clarity of | Clear solution | # | # | # |
| | Solution | essentially free from | | | |
| | | visible particulate | | | |
| | | matter | | | |
| 3. | Color and achromicity of | Color of solution | @ | @ | @ |
| | solution | should not be more | | | |
| | | intense than the USP | | | |
| | | matching fluid A | | | |
| 4. | pH | 2.5 to 3.5 | 3.38 | 3.49 | 3.36 |
| 5. | Assay of Midazolam | 90.0% to 110.0% | 98.92 | 98.76 | 98.99 |
| 6. | Organic impurity | | | | |
| | Reduced reduced | NMT 0.5% | ND | ND | 0.002 |
| | midazolam | | | | |
| | 6H-midazolam isomer | NMT 0.5% | 0.007 | 0.006 | 0.010 |
| | Any unspecified Impurity | NMT 0.1% | 0.014 | 0.034 | 0.022 |
| | Total Impurities | NMT 1.1% | 0.098 | 0.185 | 0.172 |
| 7 | Particulate matter | For >10 μm:-Not | 10 μm: 213.3 | 10 μm: 103.0 | 10 μm: 187.0 |
| | | More Than 6000 | 25 μm: 6.7 | 25 μm: 0.0 | 25 μm: 0.0 |
| | | particles per | | | |
| | | container | | | |
| | | For >25 μm:-Not | | | |
| | | More Than 600 | | | |
| | | particles per | | | |
| | | container | | | |

* Clear colorless solution
# Clear solution essentially free from visible particulate matter
@ Color of solution is less intense than the USP matching fluid A
ND: Not detected

### Example 2: Impact of Sodium Chloride on Midazolam Premix Formulations

This example provides stability testing of seven different midazolam premix formulations contained in VIAFLO bags. The seven formulations contained varying sodium chloride amounts. Results from stability testing of 0.5 mg/ml or 1 mg/ml midazolam HCl formulations in sodium chloride amounts of 0.8%, 0.855%, 0.9%, and 0.945% are provided below. pH is described in the tables (Table 5 to Table 11). Formulations were tested for 6 months at 40 degrees Celsius at no more than 25% relative humidity (RH) and at 25 degrees Celsius for 12 months at 40% RH.

1 mg/mL (100 mL VIAFLO pack)):
1. Batch with 0.800% sodium chloride (8.00 mg/ml) (Table 5)
2. Batch with 0.855% sodium chloride (8.55 mg/ml) (Table 6)
3. Batch with 0.900% sodium chloride (9.00 mg/ml) (Table 7)
4. Batch with 0.945% sodium chloride (9.45 mg/ml) (Table 8)

For 0.5 mg/mL (100 mL VIAFLO pack):
1. Batch with 0.855% sodium chloride (8.55 mg/ml) (Table 9)
2. Batch with 0.945% sodium chloride (9.45 mg/ml) (Table 10)
3. Batch with 0.900% sodium chloride (9.00 mg/ml) (Table 11)
   Stability data of the above-mentioned formulations is provided in Tables 5 to 11.

TABLE 5
Stability of 1 mg/ml in 0.8% sodium chloride

| | | | | 40° C. ± 2° C./ | 25° C. + 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.20 | 3.18 | 3.13 |
| 5. | Assay of Midazolam | 90.0% to 110.0% | 99.30 | 101.51 | 99.56 |
| 6. | Organic impurity | | | | |
| | Reduced reduced | NMT 0.5% | ND | ND | ND |
| | midazolam | | | | |
| | 6H-midazolam | NMT 0.5% | ND | ND | ND |
| | isomer | | | | |
| | Any unspecified | NMT 0.1% | ND | 0.02 | 0.01 |
| | Impurity | | | | |
| | Total Impurities | NMT 1.1% | 0.06 | 0.09 | 0.07 |

* Clear colorless solution
# Clear solution essentially free from visible particulate matter
@ Color of solution is less intense than the USP matching fluid A
ND: Not detected

TABLE 6
Stability of 1 mg/ml in 0.855% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.41 | 3.38 | 3.36 |
| 5. | Assay of Midazolam | 90.0% to 110.0% | 101.60 | 99.15 | 99.85 |
| 6. | Organic impurity | | | | |
| | Reduced reduced | NMT 0.5% | ND | ND | ND |
| | midazolam | | | | |
| | 6H-midazolam | NMT 0.5% | ND | ND | ND |
| | isomer | | | | |
| | Any unspecified | NMT 0.1% | ND | 0.01 | 0.01 |
| | Impurity | | | | |
| | Total Impurities | NMT 1.1% | 0.09 | 0.100 | 0.088 |

* Clear colorless solution
# Clear solution essentially free from visible particulate matter
@ Color of solution is less intense than the USP matching fluid A
ND: Not detected

TABLE 7
Stability of 1 mg/ml in 0.9% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 12 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.30 | 3.33 | 3.38 |
| 5. | Assay of Midazolam | 90.0% to 110.0% | 97.49 | 100.53 | 99.21 |
| 6. | Organic impurity | | | | |
| | Reduced reduced | NMT 0.5% | ND | ND | ND |
| | midazolam | | | | |
| | 6H-midazolam | NMT 0.5% | ND | 0.005 | 0.003 |
| | isomer | | | | |
| | Any unspecified | NMT 0.1% | 0.011 | 0.015 | 0.012 |
| | Impurity | | | | |
| | Total Impurities | NMT 1.1% | 0.09 | 0.106 | 0.137 |

* Clear colorless solution
# Clear solution essentially free from visible particulate matter
@ Color of solution is less intense than the USP matching fluid A
ND: Not detected

TABLE 8
Stability of 1 mg/ml in 0.945% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.29 | 3.27 | 3.26 |
| 5. | Assay of Midazolam | 90.0% to 110.0% | 101.10 | 99.59 | 100.06 |
| 6. | Organic impurity | | | | |
| | Reduced reduced | NMT 0.5% | ND | ND | ND |
| | midazolam | | | | |
| | 6H-midazolam | NMT 0.5% | ND | ND | ND |
| | isomer | | | | |
| | Any unspecified | NMT 0.1% | ND | 0.01 | 0.009 |
| | Impurity | | | | |
| | Total Impurities | NMT 1.1% | 0.093 | 0.097 | 0.096 |

* Clear colorless solution
# Clear solution essentially free from visible particulate matter
@ Color of solution is less intense than the USP matching fluid A
ND: Not detected

TABLE 9
Stability of 0.5 mg/ml in 0.855% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.45 | 3.53 | 3.53 |
| 5. | Assay of Midazolam | 90.0% to 110.0% | 99.86 | 99.79 | 99.65 |
| 6. | Organic impurity | | | | |
| | Reduced reduced | NMT 0.5% | ND | ND | ND |
| | midazolam | | | | |
| | 6H-midazolam | NMT 0.5% | 0.005 | 0.006 | 0.005 |
| | isomer | | | | |
| | Any unspecified | NMT 0.1% | 0.013 | 0.029 | 0.022 |
| | Impurity | | | | |
| | Total Impurities | NMT 1.1% | 0.115 | 0.154 | 0.143 |

* Clear colorless solution
# Clear solution essentially free from visible particulate matter
@ Color of solution is less intense than the USP matching fluid A
ND: Not detected

TABLE 10
Stability of 0.5 mg/ml in 0.945% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.30 | 3.38 | 3.43 |
| 5. | Assay of Midazolam | 90.0% to 110.0% | 100.48 | 99.26 | 99.63 |
| 6. | Organic impurity | | | | |
| | Reduced reduced | NMT 0.5% | ND | ND | ND |
| | midazolam | | | | |
| | 6H-midazolam | NMT 0.5% | 0.004 | 0.01 | 0.01 |
| | isomer | | | | |
| | Any unspecified | NMT 0.1% | 0.034 | 0.054 | 0.045 |
| | Impurity | | | | |
| | Total Impurities | NMT 1.1% | 0.191 | 0.206 | 0.202 |

* Clear colorless solution
# Clear solution essentially free from visible particulate matter
@ Color of solution is less intense than the USP matching fluid A
ND: Not detected

TABLE 11
Stability of 0.5 mg/ml in 0.9% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 12 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.55 | 3.44 | 3.34 |
| 5. | Assay of Midazolam | 90.0% to 110.0% | 97.70 | 99.73 | 99.16 |
| 6. | Organic impurity | | | | |
| | Reduced reduced | NMT 0.5% | ND | ND | ND |
| | midazolam | | | | |
| | 6H-midazolam | NMT 0.5% | 0.005 | 0.008 | 0.008 |
| | isomer | | | | |
| | Any unspecified | NMT 0.1% | 0.010 | 0.023 | 0.015 |
| | Impurity | | | | |
| | Total Impurities | NMT 1.1% | 0.106 | 0.155 | 0.145 |

* Clear colorless solution
# Clear solution essentially free from visible particulate matter
@ Color of solution is less intense than the USP matching fluid A
ND: Not detected

### Example 3: Stability of Midazolam Premix Formulations at Various pH Values

This example provides stability testing of six different midazolam premix formulations contained in VIAFLO bags. The six formulations contained 1 mg/ml or 0.5 mg/ml midazolam in 0.9% sodium chloride at different pH values (e.g., pH 2.5, pH 3.4 and pH 3.7). Results are provided below. pH is described in the tables (Table 12 to Table 17). Formulations were tested for 6 months at 40 degrees Celsius at no more than 25% relative humidity (RH) and at 25 degrees Celsius for 6 months at 40% RH.

TABLE 12
Stability of 1.0 mg/ml in 0.9% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | To be recorded | 2.66 | 2.65 | 2.64 |
| 5. | Assay of Midazolam | 90.0% to 110.0% | 101.30 | 101.83 | 100.80 |
| 6. | Organic impurity | | | | |
| | Reduced reduced | NMT 0.5% | ND | ND | ND |
| | midazolam | | | | |
| | 6H-midazolam | NMT 0.5% | ND | ND | ND |
| | isomer | | | | |
| | Any unspecified | NMT 0.1% | ND | 0.009 | 0.009 |
| | Impurity | | | | |
| | Total impurities | NMT 1.1% | 0.07 | 0.09 | 0.09 |

* Clear colorless solution
# Clear solution essentially free from visible particulate matter
@ Color of solution is less intense than the USP matching fluid A
ND: Not detected

TABLE 13
Stability of 1.0 mg/ml in 0.9% sodium chloride at pH 3.4

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 Months | 12 Months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.39 | 3.42 | 3.41 |
| 5. | Assay of Midazolam | 90.0% to 110.0% | 95.12 | 100.07 | 100.80 |
| 6. | Organic impurity | | | | |
| | Reduced reduced | NMT 0.5% | ND | ND | ND |
| | midazolam | | | | |
| | 6H-midazolam | NMT 0.5% | ND | 0.003 | 0.004 |
| | isomer | | | | |
| | Any unspecified | NMT 0.1% | 0.008 | 0.018 | 0.017 |
| | Impurity | | | | |
| | Total Impurities | NMT 1.1% | 0.083 | 0.108 | 0.136 |
| 7 | Particulate matter | For >10 μm:-Not | 10 μm: 123.3 | 10 μm: 1686.7 | 10 μm: 126.7 |
| | | More Than 6000 | 25 μm: 0.0 | 25 μm: 166.7 | 25 μm: 20.0 |
| | | particles per | | | |
| | | container | | | |
| | | For >25 μm:-Not | | | |
| | | More Than 600 | | | |
| | | particles per | | | |
| | | container | | | |

* Clear colorless solution
# Clear solution essentially free from visible particulate matter
@ Color of solution is less intense than the USP matching fluid A
ND: Not detected

TABLE 14
Stability of 1.0 mg/ml in 0.9% sodium chloride at pH 3.5

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear⁻ solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | To be recorded | 3.53 | 3.46 | 3.45 |
| 5. | Assay of Midazolam | 90.0% to 110.0% | 100.60 | 99.39 | 98.98 |
| 6. | Organic impurity | | | | |
| | Reduced reduced | NMT 0.5% | ND | ND | ND |
| | midazolam | | | | |
| | 6H-midazolam | NMT 0.5% | ND | ND | ND |
| | isomer | | | | |
| | Any unspecified | NMT 0.1% | ND | 0.008 | 0.008 |
| | Impurity | | | | |
| | Total Impurities | NMT 1.1% | 0.09 | 0.09 | 0.09 |

* Clear colorless solution
# Clear solution essentially free from visible particulate matter
@ Color of solution is less intense than the USP matching fluid A
ND: Not detected

TABLE 15
Stability of 0.5 mg/ml in 0.9% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | To be recorded | 2.62 | 2.67 | 2.55 |
| 5. | Assay of Midazolam | 90.0% to 110.0% | 96.7 | 98.8 | 98.3 |
| 6. | Organic impurity | | | | |
| | Reduced reduced | NMT 0.5% | ND | ND | ND |
| | midazolam | | | | |
| | 6H-midazolam | NMT 0.5% | 0.008 | 0.003 | 0.003 |
| | isomer | | | | |
| | Any unspecified | NMT 0.1% | 0.016 | 0.040 | 0.026 |
| | Impurity | | | | |
| | Total Impurities | NMT 1.1% | 0.136 | 0.217 | 0.187 |

* Clear colorless solution
# Clear solution essentially free from visible particulate matter
@ Color of solution is less intense than the USP matching fluid A
ND: Not detected

TABLE 16
Stability of 0.5 mg/ml in 0.9% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 Months | 6 Months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | 2.5 to 3.5 | 3.55 | 3.44 | 3.34 |
| 5. | Assay of Midazolam | 90.0% to 110.0% | 97.70 | 99.73 | 99.16 |
| 6. | Organic impurity | | | | |
| | Reduced reduced | NMT 0.5% | ND | ND | ND |
| | midazolam | | | | |
| | 6H-midazolam | NMT 0.5% | 0.005 | 0.008 | 0.008 |
| | isomer | | | | |
| | Any unspecified | NMT 0.1% | 0.010 | 0.023 | 0.015 |
| | Impurity | | | | |
| | Total Impurities | NMT 1.1% | 0.106 | 0.155 | 0.145 |

* Clear colorless solution
# Clear solution essentially free from visible particulate matter
@ Color of solution is less intense than the USP matching fluid A
ND: Not detected

TABLE 17
Stability of 0.5 mg/ml in 0.9% sodium chloride

| | | | | 40° C. ± 2° C./ | 25° C. ± 2° C./ |
|---|---|---|---|---|---|
| | | | | NMT 25% RH | 40% ± 5% RH |
| Sr. | | | | (Horizontal) | (Horizontal) |
| No. | Test Parameters | Limit | Initial | 6 months | 6 months |
| 1. | Description | A clear, colorless | * | * | * |
| | | solution filled in bag | | | |
| 2. | Clarity of | Clear solution | # | # | # |
| | Solution | essentially free from | | | |
| | | visible particulate | | | |
| | | matter | | | |
| 3. | Color and | Color of solution | @ | @ | @ |
| | achromicity of | should not be more | | | |
| | solution | intense than the USP | | | |
| | | matching fluid A | | | |
| 4. | pH | To be recorded | 3.65 | 3.63 | 3.62 |
| 5. | Assay of Midazolam | 90.0% to 110.0% | 97.7 | 99.1 | 99.5 |
| 6. | Organic impurity | | | | |
| | Reduced reduced | NMT 0.5% | ND | ND | ND |
| | midazolam | | | | |
| | 6H-midazolam | NMT 0.5% | 0.006 | 0.003 | 0.003 |
| | isomer | | | | |
| | Any unspecified | NMT 0.1% | 0.010 | 0.021 | 0.019 |
| | Impurity | | | | |
| | Total Impurities | NMT 1.1% | 0.109 | 0.182 | 0.154 |

* Clear colorless solution
# Clear solution essentially free from visible particulate matter
@ Color of solution is less intense than the USP matching fluid A
ND: Not detected

### Example 4: Visual Inspection of 1 mg/ml Midazolam Formulation at Varying pH

Observation of bulk solution containing 1 mg/ml midazolam and 0.9% sodium chloride was taken at different pH values. pH adjustment was done by using 0.1 N NaOH solution.

TABLE 18
Visual observation

| Remark | pH | Observation |
|---|---|---|
| Initial pH of bulk solution | 3.37 | Clear colorless solution |
| pH adjusted to 4.0 | 4.00 | Clear colorless solution |
| pH adjusted to 4.5 | 4.50 | Clear colorless solution |
| pH adjusted to 5.0 | 5.00 | Clear colorless solution |
| pH adjusted to 5.5 | 5.50 | Whitish precipitation observed. |

What is claimed:

1. A pharmaceutical premix formulation in a flexible plastic container, comprising from about 0.4 mg/ml to about 1.1 mg/ml midazolam hydrochloride (HCl) and sodium chloride, wherein:
the formulation is an aqueous, premix formulation and has a pH of from about 2.5 to about 3.7; and
the flexible plastic container is fabricated from a multilayer sheeting comprising polypropylene, polyamide, and polyethylene.

2. The pharmaceutical premix formulation of claim 1, comprising from about 8.5 mg/ml to about 9.5 mg/ml sodium chloride.

3. The pharmaceutical premix formulation of claim 1, comprising from about 9.0 mg/ml sodium chloride.

4. The pharmaceutical premix formulation of claim 1, comprising about 0.9% sodium chloride.

5. The pharmaceutical premix formulation of claim 1, wherein the formulation has a pH from about 2.6 to about 3.6.

6. The pharmaceutical premix formulation of claim 1, wherein the formulation has a pH from about 2.8 to about 3.4.

7. The pharmaceutical premix formulation of claim 1, wherein the formulation has a pH from about 2.50 to about 2.95.

8. The pharmaceutical premix formulation of claim 1, wherein the formulation comprises about 0.5 mg/ml midazolam HCl.

9. The pharmaceutical premix formulation of claim 1, wherein the formulation comprises about 1 mg/ml midazolam HCl.

10. The pharmaceutical premix formulation of claim 1, comprising from about 50 to about 100 mg of midazolam HCl.

11. A pharmaceutical premix formulation in a flexible plastic container, comprising:
from about 50 to about 100 mg of midazolam HCl at a concentration from about 0.5 mg/ml to about 1.0 mg/ml, and
from about 8.4 mg/ml to about 9.4 mg/ml sodium chloride, wherein:
the formulation is an aqueous, premix formulation and has a pH from about 2.5 to about 3.7; and
the flexible plastic container is fabricated from a multilayer sheeting comprising polypropylene, polyamide, and polyethylene.

12. The pharmaceutical premix formulation of claim 11, comprising about 9 mg/ml sodium chloride.

13. The pharmaceutical premix formulation of claim 11, comprising about 50 mg of midazolam HCl at a concentration of about 0.5 mg/ml, and about 0.9% sodium chloride.

14. The pharmaceutical premix formulation of claim 11, comprising 100 mg of midazolam HCl at a concentration of about 1.0 mg/ml, and about 0.9% sodium chloride.

15. The pharmaceutical premix formulation of claim 11, wherein the formulation has a pH from about 2.50 to about 2.95.

16. The pharmaceutical premix formulation of claim 1, wherein the formulation is essentially free of a preservative.

17. A pharmaceutical premix formulation in a flexible plastic container, consisting essentially of
100 mg of midazolam HCl at a concentration of about 1.0 mg/ml, and about 0.9% sodium chloride, or
about 50 mg of midazolam HCl at a concentration of about 0.5 mg/ml, and about 0.9% sodium chloride,
wherein:
the pharmaceutical premix formulation is an aqueous, premix formulation and has a pH from about 2.5 to about 3.7; and
the flexible plastic container is fabricated from a multilayer sheeting comprising polypropylene, polyamide, and polyethylene.

18. The pharmaceutical premix formulation of claim 17, wherein the formulation is stable for at least 6 months at about 25 degrees Celsius.

19. The pharmaceutical premix formulation of claim 17 wherein the flexible, plastic container has a volume of about 50 mL or about 100 mL.

20. The pharmaceutical premix formulation of claim 17, wherein the flexible, plastic container is a bag.

21. The pharmaceutical premix formulation of claim 20, wherein the inner layer of the flexible container is polyethylene.

22. A method of sedating a human subject in need thereof, the method comprising administering the pharmaceutical premix formulation of claim 1 to the human subject.

23. A method of sedating a human subject in need thereof, the method comprising:
a) obtaining a flexible plastic container which is fabricated from a multilayer sheeting comprising polypropylene, polyamide, and polyethylene, wherein the flexible plastic container comprises
a pharmaceutical premix formulation consisting essentially of
about 100 mg of midazolam HCl at a concentration of about 1.0 mg/ml, and about 0.9% sodium chloride, or about 50 mg of midazolam HCl at a concentration of about 0.5 mg/ml, and about 0.9% sodium chloride,
wherein the pharmaceutical premix formulation is an aqueous formulation and has a pH from about 2.5 to about 3.7,
b) placing the flexible plastic container on a hook or means for suspending the flexible plastic container, and
c) intravenously administering the pharmaceutical premix formulation to the human subject.

24. A method of sedating a human subject in need thereof, the method comprising intravenously administering to the human subject in need of sedation, a pharmaceutical premix formulation comprising from about 0.5 mg/ml to about 1 mg/ml midazolam HCl and from about 0.8% to about 0.9% sodium chloride, wherein the pharmaceutical premix formulation is an aqueous, premix formulation housed in a plastic flexible container which is fabricated from a multilayer sheeting comprising polypropylene, polyamide, and polyethylene.

25. The method of claim 24, wherein the pharmaceutical premix formulation comprises 50 mg midazolam HCl at a concentration of about 0.5 mg/ml or comprises 100 mg midazolam HCl at a concentration of about 1 mg/ml.
